(19) United States
(12) Patent Application Publication
(10) Pub. No.: US 2002/0107233 A1
(43) Pub. Date: Aug. 8, 2002

(54) Steriodal derivatives

(76) Inventors: Shutsung Liao, Chicago, IL (US); Ching Song, Chicago, IL (US)

Correspondence Address: Y. ROCKY TSAO, Fish & Richardson P.C., 225 Franklin Street, Boston, MA, 02110-2804, US

(21) Appl. No.: 10/072,128

(22) Filed: Feb. 8, 2002

Related U.S. Application Data
(60) Provisional application No. 60/267,493, filed on Feb. 8, 2001.

(51) Int. Cl. A61K 31/56

(52) U.S. Cl. 514/182

(57) ABSTRACT

A compound of formula (1):

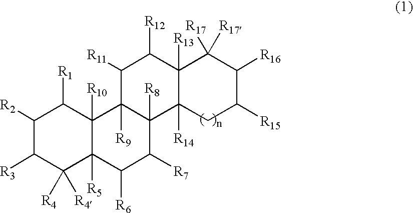

wherein each of R₁, R₂, R₄, R_{4′}, R₇, R₁₁, R₁₂, R₁₅, R₁₆, R₁₇, and R_{17′}, independently, is hydrogen, hydroxy, amino, carboxyl, oxo, halo, sulfonic acid, —O-sulfonic acid, or alkyl that is optionally inserted with —NH—, —N(alkyl)-, —O—, —S—, —SO—, —SO₂—, —O—SO₂—, —SO₂—O—, —SO₃—O—, —CO—, —CO—O—, —O—CO—, —CO—NH—, —CO—N(alkyl)-, —NH—CO—, or —N(alkyl)-CO—, and further optionally substituted with hydroxy, halo, amino, carboxyl, sulfonic acid, or —O-sulfonic acid; R₃ is X-Y—, wherein X is hydrogen, amino, carboxyl, halo, sulfonic acid, —O-sulfonic acid, or alkyl; Y is —S—, —NH—, —N(alkyl)-, —SO—, —SO₂—, —O—SO₂—, —SO₂—O—, —SO₃—O—, —CO—, —CO—O—, —O—CO—, —CO—NH—, —CO—N(alkyl)-, —NH—CO—, or —N(alkyl)-CO—; R₅ and R₆, together, are —O—; or R₅ and R₆, together, are a double bond between C-5 and C-6, and R₇ is oxo; each of R₈, R₉, R₁₀, R₁₃, and R₁₄, independently, is hydrogen, alkyl, haloalkyl, hydroxyalkyl, alkoxy, hydroxy, or amino; and n is 0, 1, or 2. Also disclosed are a method of treating hypocholesterolemia and a method of screening for an LXR agonist by administering a compound described above, a pharmaceutical composition containing at least one of the compounds described above, and an antibody against 5α, 6α-epoxycholesterol-3-sulfate or 7-ketocholesterol-3-sulfate.

### CROSS REFERENCE TO RELATED APPLICATION

Pursuant to 35 U.S.C. §119(e), this application claims the benefit of prior U.S. provisional application No. 60/267,493, filed Feb. 8, 2001.

### BACKGROUND OF THE INVENTION

Cholesterol has two primary biochemical roles: (1) as an integral component of the plasma membrane in cells, and (2) as a biosynthetic precursor in steroidogenesis in endocrine cells of the adrenal gland, ovary, testes, and placenta. Intracellular cholesterol levels are affected by de novo cholesterol synthesis, and uptake and efflux of cholesterol. Hypocholesterolemia, i.e., deficiency of cholesterol, causes diseases such as affective disorders.

Liver X receptors (LXRs), members of the nuclear receptor super-family, include LXRα and Ubiquitous Receptor (UR, also called LXRβ). Several direct target genes of LXRs are involved in cholesterol reverse transport and disposal. Examples of these genes include the CYP7A gene coding for cholesterol 7α-hydroxylase, the rate-limiting enzyme for bile acid synthesis from cholesterol, and the genes coding for cholesteryl ester transfer protein (CETP), ABC1, and ABC8. LXRs are also believed to be involved in de novo cholesterol biosynthesis.

Thus, increasing the cholesterol levels by administering an LXR antagonist, to reduce cholesterol reverse transport and disposal or to enhance de novo cholesterol biosynthesis, provides a means of treating hypocholesterolemia.

### SUMMARY OF THE INVENTION

One aspect of the present invention relates to compounds of the following formula:

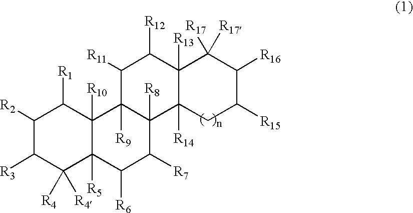

wherein each of R₁, R₂, R₄, R_{4′}, R₇, R₁₁, R₁₂, R₁₅, R₁₆, R₁₇, and R_{17′}, independently, is hydrogen, hydroxy, amino, carboxyl, oxo, halo, sulfonic acid, —O-sulfonic acid, or alkyl that is optionally inserted with —O—, —S—, —NH—, —N(alkyl)-, —SO—, —SO₂—, —O—SO₂—, —SO₂—O—, —SO₃—O—, —CO—, —CO—O—, —O—CO—, —CO—NH—, —CO—N(alkyl)-, —NH—CO—, or —N(alkyl)-CO—, and further optionally substituted with hydroxy, halo, amino, carboxyl, sulfonic acid, or —O— sulfonic acid; R₃ is X—Y—, wherein X is hydrogen, amino, carboxyl, halo, sulfonic acid, —O— sulfonic acid, or alkyl; Y is —S—, —NH—, —N(alkyl)-, —SO—, —SO₂—, —O—SO₂—, —SO₂—O—, —SO₃—O—, —CO—, —CO—O—, —O—CO—, —CO—NH—, —CO—N(alkyl)-, —NH—CO—, or —N(alkyl)-CO—; R₅ and R₆, together, are —O—; or R₅ and R₆, together, are a double bond between C-5 and C-6, and R₇ is oxo; each of R₈, R₉, R₁₀, R₁₃, and R₁₄, independently, is hydrogen, alkyl, haloalkyl, hydroxyalkyl, alkoxy, hydroxy, or amino; and n is 0, 1, or 2. The term “alkyl,” the prefix “alk” (as in alkoxy), and the suffix “-alkyl” (as in hydroxyalkyl) all refer to C₁₋₁₈ linear or branched. The term “insert” means that a substituent, e.g., R₁ or R₂, is connected to a ring carbon atom via an inserted group, e.g., —O—, —S—, or —NH— mentioned above. Unless defined otherwise, all the ring carbon atoms in formula (1) is saturated with hydrogen.

Referring to formula (1), one subset of the compounds of this invention are featured by that R₅ and R₆, together, are —O—. Another subset are featured by that R₅ and R₆, together, are a double bond between C-5 and C-6, and R₇ is oxo. Two exemplary compounds are 5α, 6α-epoxycholesterol-3-sulfate and 7-ketocholesterol-3-sulfate, two new compounds discovered in human blood and tissues.

Salts of the compounds described above, if applicable, are also within the scope of this invention. Such a salt can be formed, for example, between a compound having a carboxylate and a cationic counterion such as an alkali metal cation, e.g., a sodium ion or a potassium ion; or an ammonium cation that can be substituted with organic groups, e.g., a tetramethylammonium ion or a diisopropyl-ethylammonium ion. Such a salt can also be formed between a compound having a protonated amino group and an anionic counterion, e.g., a sulfate ion, a nitrate ion, a phosphate ion, or an acetate ion.

Compounds of this invention unexpectedly antagonize LXRs, e.g., LXRα and UR, greatly enhance de novo biosynthesis of cholesterol, and reduce reverse transport and disposal of cholesterol, thereby increasing intracellular cholesterol levels. Thus, another aspect of the present invention relates to a method of treating hypocholesterolemia. The method includes administering to a subject in need thereof an effective amount of one or more of the compounds described above.

Also within the scope of this invention is a method of evaluating a compound for its agonistic effect on an LXR with one of the above-described compounds. Further within the scope of this invention is an antibody specifically against 5α, 6α-epoxycholesterol-3-sulfate or 7-ketocholesterol-3-sulfate.

The details of several embodiments of this invention are set forth in the description below. Other features, objects, and advantages of the invention will be apparent from the description and from the claims.

### DETAILED DESCRIPTION OF THE INVENTION

A 3-sulfate compound of this invention, e.g., 5α, 6α-epoxycholesterol-3-sulfate or 7-keto-cholesterol-3-sulfate, can be prepared by first reacting triethylamine with chlorosulfonic acid to produce a triethylamine-sulfur trioxide complex. The complex is then reacted with a tetracyclic compound substituted at 3-C with hydroxy to obtain the sulfate compound. A detailed description of preparing these two compounds are provided in Examples 1 and 2, respectively.

Other compounds of this invention can be synthesized by similar methods in which other suitable reagents, instead of a triethylamine-sulfur trioxide complex, are used to react with a tetracyclic compound. Examples of such suitable reagents include (1) magnesium methyl carbonate for introducing a —(C═O)—O— linkage at 3-C, and (2) amide, triphenylphosphine, and diethyl azodicarboxylate, also for introducing —NH—C(═O)— at 3-C.

Compounds of this invention can antagonize LXRs, e.g., LXRα and UR, to reduce reverse transport and disposal of cholesterol or enhance de novo biosynthesis of cholesterol, thereby increasing intracellular cholesterol levels. Thus, another aspect of this invention relates to a method of treating hypocholesterolemia by administering to a subject in need thereof an effective amount of a compound (or its salt) of this invention. “An effective amount,” in general, refers to the amount of the compound which is required to confer a therapeutic effect on the treated subject. The interrelationship of dosages for animals and humans (based on milligrams per square meter of body surface) is described by Freireich et al., Cancer Chemother. Rep., 1966, 50, 219. Body surface area may be approximately determined from height and weight of the patient. See, e.g., Scientific Tables, Geigy Pharmaceuticals, Ardley, N.Y., 1970, 537. Effective doses will also vary, as recognized by those skilled in the art, depending on the route of administration, excipient usage, and the possibility of co-usage with other therapeutic treatments including use of other anti-hypocholesterolemia agents. An effective amount of the compound is formulated with a pharmaceutically acceptable carrier to form a pharmaceutical composition before it is administered to a subject in need of treatment of hypocholesterolemia.

The pharmaceutical composition may be administered via a parenteral route, e.g., topically, subcutaneously, intraperitoneally, intramuscularly, and intravenously. Examples of parenteral dosage forms include aqueous solutions of the active compound, in an isotonic saline, 5% glucose, or any other well-known pharmaceutically acceptable carrier. Solubilizing agents, such as cyclodextrins, or other solubilizing agents well known to those familiar with the art, can also be included in the pharmaceutical composition.

The active compound can be formulated into dosage forms for other routes of administration (e.g., orally, mucosally, percutaneously, or via inhalation) utilizing well known methods. The pharmaceutical composition can be formulated, for example, in dosage forms for oral administration in a capsule, a gel seal, or a tablet. Capsules may comprise any well known pharmaceutically acceptable material such as gelatin or cellulose derivatives. Tablets may be formulated in accordance with the conventional procedure by compressing mixtures of the active compounds, a solid carrier, and a lubricant. Examples of solid carriers include starch and sugar bentonite. The compound can also be administered in a form of a hard shell tablet or capsule containing, for example, lactose or mannitol as a binder, a conventional filler, and a tableting agent.

Also within the scope of this invention are a pharmaceutical composition containing a compound, and the use of a compound for the manufacture of a medicament for treating hypocholesterolemia.

The compounds can be preliminarily screened for their efficacy in treating hypocholesterolemia by one or more of the following in vitro assays:

The effect of a compound on antagonizing an LXR, e.g., LXRα or UR, can be assessed by an in vitro reporter gene transactivation assay. For example, kidney cells are transfected with a luciferase reporter gene (which includes a human c-fos minimal promoter) and an LXR. After incubating the transfected cells with a compound to be tested, the activity of luciferase is measured to determine the transactivation extent of the reporter gene.

The effect of a compound on antagonizing an LXR can also be assessed by an in vitro co-activator recruitment assay. For example, a fusion protein of glutathione-S-transferase (GST) and an LXR is incubated with and bound to glutathione-agarose beads. The beads are then incubated with a labeled co-activator, a compound to be tested, and, optionally, an LXR agonist. The bound protein is eluted from the beads with a buffer, and then separated on a gel for quantification, by autoradiography, of binding between the co-activator and UR.

The effect of a compound on enhancing de novo cholesterol biosynthesis can be assessed by monitoring incorporation of [2-¹⁴C]acetic acid into cholesterol in cultured cells. For example, kidney cells are seeded in a medium and incubated with a compound to be tested and labeled acetic acid. After the medium is removed from the cells, the lipids contained in the cells and the medium are extracted. Insoluble material from the extraction can be dissolved in an aqueous solution for total protein determination. The radioactivity of labeled cholesterol in the extracted lipids is measured to determine the cholesterol amount.

In vivo screening can be performed by following procedures well known in the art.

The present invention also relates to a method of screening for LXR agonists in the presence of one or more of the above-described compounds by following one of the assays described in the preceding paragraphs above. As each compound of this invention can antagonize an LXR, its use in the screening method lowers the assay background to provide a more pronounced observation of an agonistic effect. LXR agonists thus selected can be used to treat diseases related to high cholesterol levels, e.g., atherosclerosis, by reducing endogenous cholesterol levels.

The present invention further relates to a polyclonal or monoclonal antibody specifically against 5α, 6α-epoxycholesterol-3-sulfate or 7-ketocholesterol-3-sulfate. For production of the antibody, see, e.g., Harlow et al., Antibodies: A Laboratory Manual, Cold Spring Harbor Press, 1988, Cold Spring Harbor, N.Y. The antibody can be used to determine levels of endogenous 5α, 6α-epoxycholesterol-3-sulfate or 7-ketocholesterol-3-sulfate in an immunological assays such as radioimmunoassy and enzyme-linked immunoabsorbent assay. E.g., see Coligan et al., Current Protocols in Immunology, John Wiley & Sons, Inc., 1998, New York, N.Y. Abnormal levels of these compounds can be used as indicators of cholesterol-related diseases.

Without further elaboration, it is believed that one skilled in the art can, based on the description herein, utilize the present invention to its fullest extent. All publications recited herein are hereby incorporated by reference in their entirety. The following specific examples, which describe synthesis and biological testing of various compounds of the present invention, are therefore, to be construed as merely illustrative, and not limitative of the remainder of the disclosure in any way whatsoever.

### EXAMPLE 1

### Synthesis of 5α, 6α-epoxycholesterol-3-sulfate (ECHS)

To 200 mL stirred methylene chloride containing 1.0 mole triethylamie in an ice bath was added dropwise 0.5 mole chlorosulfonic acid over 2 hours. The resultant solution was briefly washed with ice-cold water, dried over anhydrous magnesium sulfate, and filtered. The filterate was concentrated to about 100 mL under a reduced pressure, heated to boiling, and added dropwise to 150 mL stirred ethyl ether to obtain a solution. The solution thus obtained was allowed to cool to room temperature and then sit at 4° C. for 4 hours to produce a crystalline triethylamine-sulfur trioxide complex.

To 1.0 mL dimethyl formamide solution containing 0.05 mmole 5α,6α-epoxy-3β-hydroxy-cholestane was added 0.55 mmole triethylamine-sulfur trioxide complex. The resultant solution was well mixed at room temperature for an hour, added with 2 drops of water, and then stirred at 40° C. for another hour. The solution was then poured into 20 mL stirred ice-cold anhydrous ethyl ether. The mixture was allowed to stand at 4° C. for 4 hours to produce crystalline ECHS.

¹H NMR (CDCl₃) δ(ppm): 0.602 (3H, s, 18-CH₃), 2.869 (1H, s, 6-H), and 4.565 (1H, m, 3-H).

### EXAMPLE 2

### Synthesis of 7-keto-cholesterol-3-sulfate (KCHS)

KCHS was prepared by following the same method described in Example 1, except that 3β-hydroxy-, Δ⁵-cholest-7-one was used, instead of 5α, 6α-epoxy-3β-hydroxy-cholestane.

### EXAMPLE 3

### Reporter Gene Transactivation Assay

Human embryonic kidney 293 cells were seeded into 48-well culture plates at 10⁵ cells per well in DMEM supplemented with 10% fetal bovine serum. After incubation for 24 hours, the cells were transfected by the calcium phosphate coprecipitation method with 250 ng of a pGL3/UREluc reporter gene that consisted of three copies of AGGTCAagccAGGTCA fused to nucleotides −56 to +109 of the human c-fos promoter in front of the firefly luciferase gene in the plasmid basic pGL3 (Promega, Madison, Wis.), 40 ng pSG5/hRXR_{\dot{E}}, 40 ng pSG5/rUR or CMX/hLXRα, 10 ng pSG5/hGrip1, 0.4 ng CMV/R-luc (transfection normalization reporter, Promega) and 250 ng carrier DNA per well. After incubation for another 12 to 24 hours, the cells were washed with phosphate buffer saline and then refed with DMEM supplemented with 4% delipidated fetal bovine serum. An ethanol solution containing a compound to be tested (i.e., ECHS triethylammonium or KCHS triethylammonium) was added in duplicate to the DMEM cell culture with the final concentration of the compound of 1 to 10 μM and the final ethanol concentration of 0.2%. After incubation for another 24 to 48 hours, the cells were harvested and the luciferase activity was measured with a commercial kit (Promega Dual luciferase II) on a Monolight luminometer (Becton Dickenson, Mountain View, Calif.). The results show that both ECHS and KCHS were potent inhibitors of the basal reporter gene transactivation by both LXRα and UR.

### EXAMPLE 4

### Co-activator Recruitment Assay

A GST-rUR fusion protein was expressed in E. coli strain BL21 using the expression plasmid pGEX (Pharmacia, Uppsala, Sweden). The cells were lysed by one cycle of freeze-thaw and sonication. The supernatant, prepared by centrifugation at 45,000×g for an hour, was incubated with glutathione-agarose for 10 minutes at 4° C. The agarose was washed with a pH 7.5 binding buffer containing HEPES (20 mM), EDTA (10 mM), Na₂MoO₄ (10 mM), é-mercaptoethanol (1 mM), DTT (1 mM), PMSF (0.5 mM), and aprotinin (2 îg/mL). After the wash, 5α-cholanoic acid methyl ester (CAM), an LXR agonist, was immediately added to a final concentration of 0.1 to 10 μM.

Human Grip1, a co-activator, was produced and labeled with [³⁵S]methionine by in vitro translation using a rabbit reticulocyte lysate. [³⁵S]Grip1-containing reticulate lysate (2 îL) was added to the GST-rUR-bound agarose beads in 100 μL binding buffer, followed by addition of an ethanol solution containing a compound to be tested (i.e., ECHS or KCHS) to a final concentration of 1 to 10 μM. The mixture was incubated at room temperature for 30 minutes. The agarose beads were then washed with the binding buffer. The bound protein was eluted with a SDS-PAGE loading buffer and then separated on a 8% SDS-PAGE gel. The gel, which contained the protein, was dried and subjected to autoradiography. The radioactivity of Grip1 was measured with a STORM phosphoimager (Molecular Dynamics, Sunnyvale, Calif.) for quantification of the co-activator recruitment. The results show that both ECHS and KCHS suppressed the co-activator recruitment.

### EXAMPLE 5

### Effect on de novo Cholesterol Biosynthesis

Macrophage J774 and kidney 293 cells were seeded in 6-well plates in a Complete™ medium (Cellgro, Mediatech Inc., Hermdon, Va.) which is free of serum, cholesterol, and cholesterol acceptors. After 24 hours, ECHS was added to the cell culture. After incubation for 24 hours, 1 mCi of [2-¹⁴C]acetic acid was added to each well. After incubation for another 24 hours, the medium was removed and lipids in the medium were extracted with chloroform/methanol (volume ratio 2:1) mixed solution. The cells attached to the plates were extracted three times with hexane/isopropanol (volume ratio 2:1) mixed solvent. Insoluble material after the extraction was first dissolved in a 1.0 N NaOH solution and used for total protein determination by the method described in Bradford, Anal. Biochem., 1976, 72:248-254. The extracted lipids were separated by thin-layer chromatography and the radioactivity of each fraction was measured by using a STORM860 phosphoimager (Molecular Dynamics, Sunnyvale, Calif.). The identity of the cholesterol fraction was confirmed by using a cholesterol standard. The results show that ECHS unexpectedly promoted de novo cholesterol synthesis by 50% to 10-fold.

### Other Embodiments

A number of embodiments of the invention have been described. Nevertheless, it will be understood that various modifications may be made without departing from the spirit and scope of the invention. For example, cholesterol levels in beef or pork can be increased by feeding cattle or swine with fodder containing a compound of this invention. In other words, a compound of this invention can be used to treat “hypocholesterolemia” (physiologically normal cholesterol levels, but regarded as too low by some gourmets) in cattle or swine, thereby increasing the cholesterol levels as is preferred by some gourmets. Accordingly, other embodiments are within the scope of the following claims.

What is claimed is:

1. A compound of formula (1): 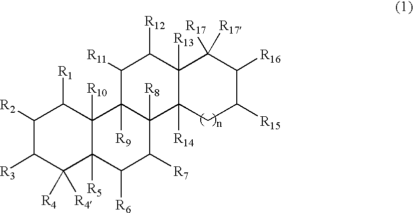
wherein
each of R₁, R₂, R₄, R_{4′}, R₇, R₁₁, R₁₂, R₁₅, R₁₆, R₁₇, and R_{17′}, independently, is hydrogen, hydroxy, amino, carboxyl, oxo, halo, sulfonic acid, —O-sulfonic acid, or alkyl that is optionally inserted with —NH—, —N(alkyl)-, —O—, —S—, —SO—, —SO₂—, —O—SO₂—, —SO₂—O—, —SO₃—O—, —CO—, —CO—O—, —O—CO—, —CO—NH—, —CO—N(alkyl)-, —NH—CO—, or —N(alkyl)-CO—, and further optionally substituted with hydroxy, halo, amino, carboxyl, sulfonic acid, or —O-sulfonic acid;
R₃ is X-Y—, wherein X is hydrogen, amino, carboxyl, halo, sulfonic acid, —O-sulfonic acid, or alkyl; Y is —S—, —NH—, —N(alkyl)-, —SO—, —SO₂—, —O—SO₂—, —SO₂—O—, —SO₃—O—, —CO—, —CO—O—, —O—CO—, —CO— NH—, —CO—N(alkyl)-, —NH—CO—, or —N(alkyl)-CO—;
R₅ and R₆, together, are —O—; or R₅ and R₆, together, are a double bond between C-5 and C-6, and R₇ is oxo;
each of R₈, R₉, R₁₀, R₁₃, and R₁₄, independently, is hydrogen, alkyl, haloalkyl, hydroxyalkyl, alkoxy, hydroxy, or amino; and
n is 0, 1, or 2.

2. The compound of claim 1, wherein X is hydrogen or amino, and Y is —O—SO₂—, —SO₂—O—, —SO₃—O—, —CO—, —CO—O—, —O—CO—, —CO—NH—, —CO—N(alkyl)-, —NH—CO—, or —N(alkyl)-CO—.

3. The compound of claim 1, wherein R₅ and R₆, together, are —O—.

4. The compound of claim 3, wherein X is hydrogen or amino, and Y is —O—SO₂—, —SO₂—O—, —SO₃—O—, —CO—, —CO—O—, —O—CO—, —CO—NH—, —CO—N(alkyl)-, —NH—CO—, or —N(alkyl)-CO—.

5. The compound of claim 4, wherein X is hydrogen, and Y is —SO₃.

6. The compound of claim 3, wherein —O— is on the α side of C-5 and C-6.

7. The compound of claim 6, wherein X is hydrogen or amino, and Y is —O—SO₂—, —SO₂—O—, —SO₃—O—, —CO—, —CO—O—, —O—CO—, —CO—NH—, —CO—N(alkyl)-, —NH—CO—, or —N(alkyl)-CO—.

8. The compound of claim 7, wherein X is hydrogen, and Y is —SO₃.

9. The compound of claim 8, wherein R₁, R₂, R₄, R_{4′}, R₇, R₈, R₉, R₁₁, R₁₂, R₁₄, R₁₅, R₁₆, and R₁₇ are hydrogen; and each of R₁₀, R₁₃, and R_{17′}, independently, is alkyl.

10. The compound of claim 9, wherein the compound is 5α, 6α-epoxycholesterol-3-sulfate.

11. An antibody which is specifically against the compound of claim 10.

12. The compound of claim 1, wherein R₅ and R₆, together, are a double bond between C-5 and C-6, and R₇ is oxo.

13. The compound of claim 12, wherein X is hydrogen or amino, and Y is —O—SO₂—, —SO₂—O—, —SO₃—O—, —CO—, —CO—O—, —O—CO—, —CO—NH—, —CO—N(alkyl)-, —NH—CO—, or —N(alkyl)-CO—.

14. The compound of claim 13, wherein X is hydrogen, and Y is —SO₃—O—.

15. The compound of claim 14, wherein R₁, R₂, R₄, R_{4′}, R₇, R₈, R₉, R₁₁, R₁₂, R₁₄, R₁₅, R₁₆, and R₁₇ are hydrogen; and each of R₁₀, R₁₃, and R_{17′}, independently, is alkyl.

16. The compound of claim 15, wherein the compound is 7-keto-cholesterol-3-sulfate.

17. An antibody which is specifically against the compound of claim 16.

18. A method of treating hypocholesterolemia, comprising administering to a subject in need thereof an effective amount of a compound of formula (1): 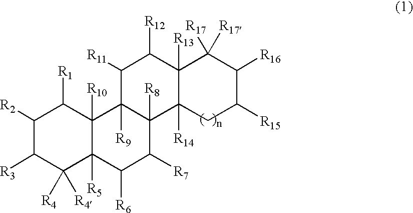
wherein
each of R₁, R₂, R₄, R_{4′}, R₇, R₁₁, R₁₂, R₁₅, R₁₆, R₁₇, and R_{17′}, independently, is hydrogen, hydroxy, amino, carboxyl, oxo, halo, sulfonic acid, —O-sulfonic acid, or alkyl that is optionally inserted with —O—, —S—, —NH—, —N(alkyl)-, —SO—, —SO₂—, —O—SO₂—, —SO₂—O—, —SO₃—O—, —CO—, —CO—O—, —O—CO—, —CO—NH—, —CO—N(alkyl)-, —NH—CO—, or —N(alkyl)-CO—, and further optionally substituted with hydroxy, halo, amino, carboxyl, sulfonic acid, or —O-sulfonic acid;
R₃ is X-Y—, wherein X is hydrogen, amino, carboxyl, halo, sulfonic acid, —O-sulfonic acid, or alkyl; Y is —S—, —NH—, —N(alkyl)-, —SO—, —SO₂—, —O—SO₂—, —SO₂—O—, —SO₃—O—, —CO—, —CO—O—, —O—CO—, —CO—NH—, —CO—N(alkyl)—, —NH—CO—, or —N(alkyl)-CO—;
R₅ and R₆, together, are —O—; or R₅ and R₆, together, are a double bond between C-5 and C-6, and R₇ is oxo;
each of R₈, R₉, R₁₀, R₁₃, and R₁₄, independently, is hydrogen, alkyl, haloalkyl, hydroxyalkyl, alkoxy, hydroxy, or amino; and
n is 0, 1, or 2.

19. The method of claim 18, wherein X is hydrogen or amino, and Y is —O—SO₂—, —SO₂—O—, —SO₃—O—, —CO—, —CO—O—, —O—CO—, —CO—NH—, —CO—N(alkyl)-, —NH—CO—, or —N(alkyl)-CO—.

20. The method of claim 18, wherein R₅ and R₆, together, are —O—.

21. The method of claim 20, wherein X is hydrogen or amino, and Y is —O—SO₂—, —SO₂—O—, —SO₃—O—, —CO—, —CO—O—, —O—CO—, —CO—NH—, —CO—N(alkyl)-, —NH—CO—, or —N(alkyl)-CO—.

22. The method of claim 21, wherein X is hydrogen, and Y is —SO₃—O—.

23. The method of claim 20, wherein —O— is on the α side of C-5 and C-6.

24. The method of claim 23, wherein X is hydrogen or amino, and Y is —O—SO₂—, —SO₂—O—, —SO₃—O—, —CO—, —CO—O—, —O—CO—, —CO—NH—, —CO—N(alkyl)-, —NH—CO—, or —N(alkyl)-CO.

25. The method of claim 24, wherein X is hydrogen, and Y is —SO₃—O—.

26. The method of claim 25, wherein R₁, R₂, R₄, R_{4′}, R₇, R₈, R₉, R₁₁, R₁₂, R₁₄, R₁₅, R₁₆, and R₁₇ are hydrogen, and each of R₁₀, R₁₃, and R_{17′}, independently, is alkyl.

27. The method of claim 26, wherein the compound is 5α, 6α-epoxycholesterol-3-sulfate.

28. The method of claim 18, wherein R₅ and R₆, together, are a double bond between C-5 and C-6, and R₇ is oxo.

29. The method of claim 28, wherein X is hydrogen or amino, and Y is —O—SO₂—, —SO₂—O—, —SO₃—O—, —CO—, —CO—O—, —O—CO—, —CO—NH—, —CO—N(alkyl)-, —NH—CO—, or —N(alkyl)-CO—.

30. The method of claim 29, wherein X is hydrogen, and Y is —SO₃—O—.

31. The method of claim 30, wherein R₁, R₂, R₄, R_{4′}, R₇, R₈, R₉, R₁₁, R₁₂, R₁₄, R₁₅, R₁₆, and R₁₇ are hydrogen, and each of R₁₀, R₁₃, and R_{17′}, independently, is alkyl.

32. The method of claim 31, wherein the compound is 7-keto-cholesterol-3-sulfate.

33. A pharmaceutical composition comprising a compound of formula (1): 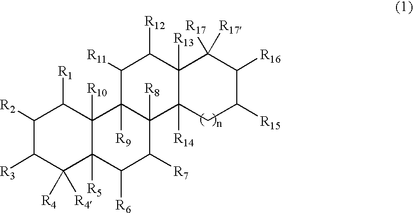
wherein
each of R₁, R₂, R₄, R_{4′}, R₇, R₁₁, R₁₂, R₁₅, R₁₆, R₁₇, and R_{17′}, independently, is hydrogen, hydroxy, amino, carboxyl, oxo, halo, sulfonic acid, —O-sulfonic acid, or alkyl that is optionally inserted with —O—, —S—, —NH—, —N(alkyl)-, —SO—, —SO₂—, —O—SO₂—, —SO₂—O—, —SO₃—O—, —CO—, —CO—O—, —O—CO—, —CO—NH—, —CO—N(alkyl)-, —NH—CO—, or —N(alkyl)-CO—, and further optionally substituted with hydroxy, halo, amino, carboxyl, sulfonic acid, or —O-sulfonic acid;
R₃ is X-Y—, wherein X is hydrogen, amino, carboxyl, halo, sulfonic acid, —O-sulfonic acid, or alkyl; Y is —S—, —NH—, —N(alkyl)-, —SO—, —SO₂—, —O—SO₂—, —SO₂—O—, —SO₃—O—, —CO—, —CO—O—, —O—CO—, —CO—NH—, —CO—N(alkyl)-, —NH—CO—, or —N(alkyl)-CO—;
R₅ and R₆, together, are —O—; or R₅ and R₆, together, are a double bond between C-5 and C-6, and R₇ is oxo;
each of R₈, R₉, R₁₀, R₁₃, and R₁₄, independently, is hydrogen, alkyl, haloalkyl, hydroxyalkyl, alkoxy, hydroxy, or amino; and
n is 0, 1, or 2;
and a pharmaceutically acceptable carrier.

34. The composition of claim 33, wherein X is hydrogen or amino, and Y is —O—SO₂—, —SO₂—O—, —SO₃—O—, —CO—, —CO—O—, —CO—, —CO—NH—, —CO—N(alkyl)-, —NH—CO—, or —N(alkyl)-CO—.

35. The composition of claim 33, wherein R₅ and R₆, together, are —O—.

36. The composition of claim 35, wherein X is hydrogen or amino, and Y is —O—SO₂—, —SO₂—O—, —SO₃—O—, —CO—, —CO—O—, —O—CO—, —CO—NH—, —CO—N(alkyl)-, —NH—CO—, or —N(alkyl)-CO—.

37. The composition of claim 36, wherein X is hydrogen, and Y is —SO₃—O—.

38. The composition of claim 35, wherein —O— is on the a side of C-5 and C-6.

39. The composition of claim 38, wherein X is hydrogen or amino, and Y is —O—SO₂—, —SO₂—O—, —SO₃—O—, —CO—, —CO—O—, —O—CO—, —CO—NH—, —CO—N(alkyl)-, —NH—CO—, or —N(alkyl)-CO—.

40. The composition of claim 39, wherein X is hydrogen, and Y is —SO₃—O—.

41. The composition of claim 40, wherein R₁, R₂, R₄, R_{4′}, R₇, R₈, R₉, R₁₁R₁₂, R₁₄, R₁₅, R₁₆, and R₁₇ are hydrogen, and each of R₁₀, R₁₃, and R_{17′}, independently, is alkyl.

42. The composition of claim 41, wherein the compound is 5α, 6α-epoxycholesterol-3-sulfate.

43. The composition of claim 33, wherein R₅ and R₆, together, are a double bond between C-5 and C-6, and R₇ is oxo.

44. The composition of claim 33, wherein X is hydrogen or amino, and Y is —O—SO₂—, —SO₂—O—, —SO₃—O—, —CO—, —CO—O—, —O—CO—, —CO—NH—, —CO—N(alkyl)-, —NH—CO—, or —N(alkyl)-CO—.

45. The composition of claim 44, wherein X is hydrogen, and Y is —SO₃—O—.

46. The composition of claim 45, wherein R₁, R₂, R₄, R_{4′}, R₇, R₈, R₉, R₁₁, R₁₂, R₁₄, R₁₅, R₁₆, and R₁₇ are hydrogen, and each of R₁₀, R₁₃, and R_{17′}, independently, is alkyl.

47. The composition of claim 46, wherein the compound is 7-keto-cholesterol-3-sulfate.

48. A method of evaluating a compound for its agonistic effect on an liver X receptor, comprising:
contacting the compound to be evaluated with the liver X receptor in the presence of a compound of formula (1): 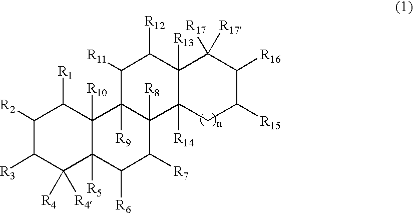
wherein
each of R₁, R₂, R₄, R_{4′}, R₇, R₁₁, R₁₂, R₁₅, R₁₆, R₁₇, and R_{17′}, independently, is hydrogen, hydroxy, amino, carboxyl, oxo, halo, sulfonic acid, —O-sulfonic acid, or alkyl that is optionally inserted with —O—, —S—, —NH—, —N(alkyl)-, —SO—, —SO₂—, —O—SO₂—, —SO₂—O—, —SO₃—O—, —CO—, —CO—O—, —O—CO—, —CO—NH—, —CO—N(alkyl)-, —NH—CO—, or —N(alkyl)-CO—, and further optionally substituted with hydroxy, halo, amino, carboxyl, sulfonic acid, or —O-sulfonic acid;
R₃ is X-Y—, wherein X is hydrogen, amino, carboxyl, halo, sulfonic acid, —O-sulfonic acid, or alkyl; Y is —S—, —NH—, —N(alkyl)-, —SO—, —SO₂—, —O—SO₂—, —SO₂—O—, —SO₃—O—, —CO—, —CO—O—, —O—CO—, —CO—NH—, —CO—N(alkyl)-, —NH—CO—, or —N(alkyl)-CO—;
R₅ and R₆, together, are —O—; or R₅ and R₆, together, are a double bond between C-5 and C-6, and R₇ is oxo;
each of R₈, R₉, R₁₀, R₁₃, and R₁₄, independently, is hydrogen, alkyl, haloalkyl, hydroxyalkyl, alkoxy, hydroxy, or amino; and
n is 0, 1, or 2; and assessing the agonistic effect of the compound to be evaluated on the liver X receptor.

49. The method of claim 48, wherein X is hydrogen or amino, and Y is —O—SO₂—, —SO₂—O—, —SO₃—O—, —CO—, —CO—O—, —O—CO—, —CO—NH—, —CO—N(alkyl)-, —NH—CO—, or —N(alkyl)-CO—.

50. The method of claim 48, wherein R₅ and R₆, together, are —O—.

51. The method of claim 50, wherein X is hydrogen or amino, and Y is —O—SO₂—, —SO₂—O—, —SO₃—O—, —CO—, —CO—O—, —O—CO—, —CO—NH—, CO—N(alkyl)-, —NH—CO—, or —N(alkyl)-CO—.

52. The method of claim 51, wherein X is hydrogen, and Y is —SO₃—O—.

53. The method of claim 50, wherein —O— is on the α side of C-5 and C-6.

54. The method of claim 51, wherein X is hydrogen or amino, and Y is —O—SO₂—, —SO₂—O—, —SO₃—O—, —CO—, —CO—O—, —O—CO—, —CO—NH—, —CO—N(alkyl)-, —NH—CO—, or —N(alkyl)-CO—.

55. The method of claim 54, wherein X is hydrogen, and Y is —SO₃—O—.

56. The method of claim 55, wherein R₁, R₂, R₄, R_{4′}, R₇, R₈, R₉, R₁₁, R₁₂, R₁₄, R₁₅, R₁₆, and R₁₇ are hydrogen, and each of R₁₀, R₁₃, and R_{17′}, independently, is alkyl.

57. The method of claim 56, wherein the compound is 5α, 6α-epoxycholesterol-3-sulfate.

58. The method of claim 48, wherein R₅ and R₆, together, are a double bond between C-5 and C-6, and R₇ is oxo.

59. The method of claim 48, wherein X is hydrogen or amino, and Y is —O—SO₂—, —SO₂—O—, —SO₃—O—, —CO—, —CO—O—, —O—CO—, —CO—NH—, —CO—N(alkyl)-, —NH—CO—, or —N(alkyl)-CO—.

60. The method of claim 59, wherein X is hydrogen, and Y is —-SO₃—O—.

61. The method of claim 60, wherein R₁, R₂, R₄, R_{4′}, R₇, R₈, R₉, R₁₁, R₁₂, R₁₄, R₁₅, R₁₆, and R₁₇ are hydrogen, and each of R₁₀, R₁₃, and R_{17′}, independently, is alkyl.

62. The method of claim 61, wherein the compound is 7-keto-cholesterol-3-sulfate.
